(19) United States
(12) Patent Application Publication
(10) Pub. No.: US 2021/0275810 A1
(43) Pub. Date: Sep. 9, 2021

(54) USE OF A NEUROMODULATION SYSTEM

(71) Applicant: ONWARD MEDICAL B.V., Eindhoven (NL)

(72) Inventor: Miroslav CABAN, Renens (CH)

(73) Assignee: ONWARD MEDICAL B.V., Eindhoven (NL)

(21) Appl. No.: 17/249,516

(22) Filed: Mar. 3, 2021

(30) Foreign Application Priority Data
Mar. 4, 2020 (EP) 20160841.1

(51) Int. Cl. A61N 1/36

(52) U.S. Cl. CPC A61N 1/36062; A61N 1/36103

(57) ABSTRACT

A neuromodulation system can be used to treat a subject with at least one abnormal body function and/or disease comprising the treatment and/or modulation of at least one condition and/or function and/or dysfunction of the subject's gastrointestinal system, cardiovascular system, sensory system, urinary system, respiratory system, reproductive system, thermoregulation system and/or locomotor system.

### TECHNICAL FIELD

The present invention relates to the use of a neuromodulation system for treating a subject, in particular for treating a subject with abnormal body function and/or disease.

### BACKGROUND

A wide variety of mental and/or physical processes are controlled or influenced by neural activity. Many problems or abnormalities with body functions can be caused by dysfunction, damage, disease and/or disorders in the nervous system and/or nerves. Effectively treating such abnormalities may be very difficult.

Neuromodulation systems have been developed for the treatment of dysfunction, damage, disease and/or disorders of the nervous system. One example are neuromodulation systems for the treatment of motoric dysfunction after spinal cord injury (SCI). Such systems can deliver adaptive electrical spinal cord stimulation to facilitate and restore locomotion after neuromotor impairment. Such systems can be closed loop and can provide real-time control of epidural electrical stimulation. Such a system can include means for applying to a subject neuromodulation with adjustable stimulation parameters. These means can be operatively connected to a real-time monitoring component comprising sensors continuously acquiring feedback signals from said subject. The feedback signals can provide features of motion of the subject. The real-time monitoring component can be operatively connected to a signal processing device that receives the feedback signals and operates real-time automatic control algorithms. Such systems can improve consistency of walking in a subject with a neuromotor impairment.

As an additional example, a system can provide selective spatiotemporal electrical neurostimulation of the spinal cord. A signal processing device can receive signals from a subject and operate signal-processing algorithms to elaborate stimulation parameter settings. The device can be operatively connected to an Implantable Pulse Generator (IPG). The IPG can receive stimulation parameter settings from the signal processing device. The IPG can be configurable to simultaneously deliver independent current or voltage pulses to one or more multiple electrode arrays. The electrode arrays can be operatively connected to one or more multi-electrode arrays. These arrays can be designed and adapted for implantation at a location covering at least a portion of the spinal cord of a subject. The IPG can apply a selective spatiotemporal stimulation of the spinal circuits and/or dorsal roots. The stimulation can be multipolar stimulation. Such system can allow effective control of locomotor functions in a subject in need thereof by stimulating the spinal cord, in particular the dorsal roots, with spatiotemporal selectivity.

### DETAILED DESCRIPTION

Reference will now be made in detail to exemplary embodiments. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Neuromodulation systems can be used for the treatment of dysfunctions, damage, or disorders of the nervous system. Such dysfunctions, damage, or disorders can arise from spinal cord injury or any other disease. For example, neuromodulation systems may be used for the treatment of autonomic dysfunction after SCI (or other symptoms occurring in other diseases). The disclosed systems and methods can improve the use of a neuromodulation system for a subject with at least one impaired body function.

The disclosed embodiments can use a neuromodulation system for treating a subject, with the features described herein. In some embodiments, a neuromodulation system can be used to treating a subject with at least one abnormal body function or disease. As described herein, abnormal body function may be or may comprise any type of pathophysiological condition.

The system can perform a treatment or modulation of at least one of a condition, function, or dysfunction of the subject's gastrointestinal system, cardiovascular system, sensory system, urinary system, respiratory system, reproductive system, thermoregulation system and/or locomotor system. In particular, the specific use of a neuromodulation system for treating a subject may enable that the neuromodulation provided by a neuromodulation system is specifically adapted to at least one of a subject's needs, impairment, rehabilitation status, or environment. This may enable advancing progress in rehabilitation and thus a most efficient and fast rehabilitation process.

In general, the gastrointestinal system of the subject may comprise the upper gastrointestinal tract and/or the lower gastrointestinal tract. In particular, the gastrointestinal system may comprise the mouth, esophagus, stomach and/or intestine of the subject. In particular, the intestine may comprise the small intestine (duodenum, jejunum, ileum) and/or the large intestine (cecum, appendix, ascending colon, right colic flexure, transverse colon, left colic flexure, descending colon, sigmoid colon, rectum and/or anus). In general, sphincters of the respective organs may be included in the gastrointestinal system. Further, the gastrointestinal system of the subject may comprise the liver, gallbladder, pancreas, pancreatic duct and/or immune system of the gastrointestinal system of the subject.

The reproductive system of the subject may comprise testes, scrotum, prostate, penis, urethra, vas deferens, Cowper's gland, vulva, ovaries, breasts and/or cervix of the subject.

The urinary system of the subject may comprise kidney(s), renal pelvis, ureter, urinary bladder, urethra and/or adrenal gland(s) of the subject.

The cardiovascular system of the subject may comprise arteries, capillaries, veins, portal veins, the heart and/or coronary vessels.

The respiratory system of the subject may comprise nose, nasal cavities, sinuses, pharynx, larynx (at least partially), trachea, lung, bronchi, bronchioles and/or alveoli. The sensory system of the subject may comprise sensory neurons, sensory receptor cells, neural pathways and/or parts of the brain involved in sensory perception.

The locomotor system of the subject may comprise the skeleton of the subject and/or at least one muscle, nerve, bone, cartilage and/or tendon. In particular, the locomotor system may comprise upper and lower body parts, including arms, legs, trunk, abdomen, neck, head, etc.

The thermoregulation system of the subject may comprise sweat glands, vascularization (in particular skin vascularization), adipose tissue (in particular white adipose tissue and/or brown adipose tissue), muscles, central nervous system (e.g., Lissauer's tract, spinal cord, brain, e.g., hypothalamus, thalamus) and/or thermoreceptors.

Consistent with disclosed embodiments, the neuromodulation system may be or may comprise at least one of a neuromodulator, a controller, a processor, a sensor, a communication module, a telemetry module and/or a storage means. In particular, the neuromodulator may be a neurostimulator. The neurostimulator may be or may comprise a pulse generator (e.g., an implantable pulse generator and/or non-implantable pulse generator), a lead and/or at least one electrode.

The at least one condition, function, or dysfunction may comprise at least one of acetylcholine dysregulation, acidity of stomach, autonomic dysreflexia, back pain, sphincter function, blood volume dysregulation, breathing deficiency, bronchial tree obstruction, cardiac activity, cholesterol imbalance, motor deficits (e.g., motor deficits being or including a deficit of the motor function of at least one of the upper extremities, lower extremities, trunk, abdomen, neck, or head), mucosa renewal (e.g., mucosa renewal of the intestine, colon and/or stomach), immune system deficit, immobility of the stomach and/or intestine and/or colon, lipogenesis/lipolysis imbalance, lower limb paralysis and/or upper limb paralysis (bilateral and/or unilateral), detrusor muscle, bronchial deficiency, alveolar deficiency, mean venous pressure, mean arterial pressure, mucous layer of stomach, erectile dysfunction, neck stabilization, odor, hypotension, orthostatic hypotension, hypertension, salivary glands function, supraspinal cardiovascular regulation, swallowing, taste, thermoregulation, trunk stabilization, vision, cutaneous vascularization, sweat glands, lipid metabolism and/or glycogen metabolism. In particular, the specific use of a neuromodulation system for treating at least one of these conditions and/or functions and/or dysfunctions may enable that physiological deficits of an impaired subject are at least partially restored. In particular, this may enable that the subject's performance and/or quality of live is enhanced.

As used herein, a sphincter may be or may include any autonomous or voluntary regulated sphincter. Without limitation, the term “sphincter” may refer to the upper or lower sphincter of the esophagus, pyloric, ileocecal, urethral (e.g., lower urinary or upper urinary sphincter), or internal or external anal sphincter. Sphincter dysfunction may be or may comprise dysfunction of such a sphincter.

The at least one abnormal body function or disease may be or may comprise myasthenia, dystonia, epilepsy, muscular hyperfatigability, ulcers, Helicobacter Pylori infection, inflammatory bowel disease, Crohn's disease, ulcerative colitis, irritable bowel syndrome, spinal cord injury, incontinence (e.g., incontinence being or including complete fecal or urinary incontinence), hypertension, hypotension, sudden episodic high blood pressure, dyspnea, chronic obstructive pulmonary disease, cardiac failure, cardiac dysrhythmia, bradycardia, tachycardia, cardiac arrest, impairment of genital plexus (e.g. after prostate removal), HIV infection, herniated disk, tumor, obesity, gastroesophageal reflux disease, lower esophageal sphincter failure, achalasia, amyotrophic lateral sclerosis, trauma, stroke, damaged dorsal root, pernicious anemia, vitamin D deficiency, multiple sclerosis, diabetes mellitus, autoimmune disease, Sjogren syndrome, Mikulicz syndrome, loss of sympathetic control, dysphagia, overreactive bladder syndrome, hyperhomocysteinemia, neurodegenerative disease (e.g., neurodegenerative disease being or including Alzheimer's disease or Parkinson's disease), essential tremor, schizophrenia, osteoporosis and/or hyperthyroidism.

Consistent with disclosed embodiments, the specific use of a neuromodulation system for treating a subject at least one of these diseases or abnormal functions may enable that the symptoms of these abnormal functions or diseases to be at least partially cured (or symptoms may be relieved during stimulation). In particular, the specific use of the neuromodulation system may enhance the subject's performance or quality of life.

Consistent with disclosed embodiments, treating a subject may be or may comprise treating at least one target. A target can be or include a muscular cholinergic synapse, stomach, sympathetic and/or parasympathetic nervous system, heart, cardiac muscle, spinal cord, lower urinary sphincter, autonomous and/or voluntary regulated sphincters, Renin Angiotensin system, kidney, lower bowel sphincter, diaphragm, intercostal muscle, bronchia, smooth muscles of the pulmonary system, solitary tract, sinoatrial/atrioventricular nodes, pancreas, genital plexus, dorsal root to muscle (e.g. pharynx), colon, vago-vagal reflex, cajal cell, muscle, smooth muscle, nerve, concerned nerve or group of nerves, concerned plexus (including but not limited to genital, Auerbach, Meissner, cardiac, pulmonary, cervical, lumbar, sacral and/or celiac plexus), liver, adrenal gland, lower esophageal sphincter, alveoli, pulmonary circulation, associated cortex and/or regulatory center, salivary gland and/or detrusor muscle. In other words, a more or less specific neuromodulation may be enabled.

In some embodiments, such targets could be treated directly and/or indirectly. In other words, the at least one target may be directly the place of dysfunction and/or a way to modulate the dysfunction. In particular, conditions could be modulated by targeting these targets. In particular, intercostal muscles may include innermost, internal and/or external intercostal muscles. In particular, intercostal muscles may include innermost, internal and/or external intercostal muscles. Respiratory tractus may include respiratory system and/or associated blood circulation and/or smooth muscles. Spinal cord may include dorsal roots, afferent and/or efferent nerves, ganglions.

The use of the neuromodulation system according to the present invention may comprise the use of a neuromodulation system throughout rehabilitation of the subject and/or during at least one stage of rehabilitation. In particular, the stage of rehabilitation may be or may comprise at least one of stabilization in bed, mobilization in bed, mobilization at bedside, verticalization, blood pressure lowering, blood pressure rehabilitation, blood pressure stabilization, cardiac rehabilitation, cardiac stabilization, standing, stepping, balance training, locomotor training, fluent locomotion, pre-surgery, post-surgery, pre-implantation, post-implantation, post-setting of neuromodulation system, spinal shock and/or daily life activities. In particular, the use of the neuromodulation system may comprise the specific use of the neuromodulation system during least one specific rehabilitation stage. This may enable best possible treatment during each stage and/or phase of rehabilitation.

It is generally possible that the use of the neuromodulation system is specifically adapted to the subject's individual needs and/or preferences and/or rehabilitation stage. It is further generally possible, that the use of the neuromodulation system is altered throughout rehabilitation.

Further, the use of the neuromodulation system may comprise or may be combined with the use of an assistive device, wherein the assistive device may be or may comprise at least one of a breathing device, pulling handles, cables from ceiling, aquatic equipment, a treadmill, an exoskeleton, an orthosis, a robot, a bike, a breather, crutches, a cuff, a cuff for functional electrical stimulation, a smartwatch, a motorized mattress, non-invasive ventilation, a standing frame, a rollator, a body weight support system, elastic bands, weights, a slide board, a step machine, a trike, a walker, a robot-physiotherapist, balls and/or half-balls. In particular, the use of an assistive device together with a neuromodulation system and/or as part of a neuromodulation system may enable most efficient progress in rehabilitation. Aquatic equipment may be or may comprise at least on of noodles, floating vests, boards, etc.

The neuromodulation system may be neurostimulation system, wherein the neurostimulation system may be or may comprise an electrical neurostimulation system and/or mechanical neurostimulation system and/or pharmacological neurostimulation system. It may be possible that the pharmacological neurostimulation system comprises a drug pump. Alternatively, the neurostimulation system may be or may comprise an optical stimulation system, an ultrasound stimulation system and/or a magnetic field stimulation system.

In some embodiments, an electrical stimulation system may be or may comprise a pulse generating system. In particular, the pulse generating system may be an implantable pulse generating system (IPG). The electrical stimulation system may alternatively and/or additionally comprise an electrode array comprising multiple electrodes and/or a lead.

In some embodiments, the neuromodulation system may be or may comprise at least one of a transcutaneous electrical stimulation system (TENS), epidural electrical stimulation system (EES), functional electrical stimulation system (FES), intramuscular stimulation system (IMS), dorsal root ganglion stimulation system, subdural stimulation system, cardiac stimulation system, optogenetics, optotrodes, patch-clamp, intra-cellular electrodes and/or extra-cellular electrodes. It may be generally possible that the neuromodulation system is a central nervous system (CNS) stimulation system and/or peripheral nervous system stimulation (PNS) system.

In some embodiments, for the use of a neuromodulation system for stimulation of the CNS may comprise that neurostimulation may be applied to any area of the brain and/or spinal cord (including but not limited to spinal nerves, ganglions, nerve roots) and/or nervous plexus and/or glands related to the nervous system (e.g., medulla). In particular, electrical neurostimulation may be applied to at least one of T6-T9, T6 or higher, T2-L1, L1-L2, S2-S4, T10-L1, T10-L2 and S2-S4, C3-05, T1-T5, T5-T9, L5-S5, L2-S1, L1-S1, T7-T12, T5-T8, L2-S2, T1-T6, T1-T5, T6-T9, C1-C3, above C1, C1-C4, C1-C4 and above, N.X, N. IX, C1-C5, T7, hypothalamus, medulla, T7-L1, C1-S1, L3-S4.

Epidural Electrical Stimulation (EES) is known to restore motor control in animal and human models and has more particularly been shown to restore locomotion after spinal cord injury by artificially activating the neural networks responsible for locomotion below the spinal cord lesion. EES does not directly stimulate motor-neurons but the afferent sensory neurons prior to entering into the spinal cord. In this way, the spinal networks responsible for locomotion are recruited indirectly via those afferents, restoring globally the locomotion movement by activating the required muscle synergies.

Peripheral Nervous System (PNS) Stimulation systems used to date in the clinic are known as Functional Electrical Stimulation (FES) that provides electrical stimulation to target muscles with surface electrodes, either directly through stimulation of their motor fibers (neuro-muscular stimulation), or through a limited set reflexes (practically limited to the withdrawal reflex) or by transcutaneously stimulating the peripheral nerves. The resulting muscle fatigue has rendered FES unsuitable for use in daily life. Furthermore, successes have remained limited through cumbersome setups when using surface muscle stimulation, unmet needs in terms of selectivity (when using transcutaneous nerve stimulation) and a lack of stability (impossible to reproduce exact electrode placement on a daily basis when stimulating muscles, moving electrodes due to clothes, sweating).

A neuromodulation system consistent with disclosed embodiments may be an open-loop system and/or closed-loop system and/or pseudo-open-loop system. In some embodiments, an open-loop system may be operated manually, with a simple design. Thus, open loop systems may be stable and economical compared to closed loop systems. In some embodiments, a closed-loop system may be accurate even in the presence of non-linearities. The sensitivity of the system may be made small to make the system more stable. The closed loop system may be less affected by noise.

In some embodiments, the closed-loop system may comprise at least one sensor. In particular, this may enable that the patient's status and/or performance may be assessed and/or provided to the neuromodulation system. This may further enable that the neuromodulations performance is specifically adapted to the patient's needs. The sensor may be at least one of an accelerometer, pressure sensor, motion capture sensor, force plate, rehabilitation device, electrode, electrode lead, thermometer (e.g. nose thermometer, skin thermometer, implanted thermometer), infrared camera, doppler ultrasound, electrocardiogram, GPS, smartphone, smartwatch, action-camcorder, video processing, virtual reality module, magnetic resonance imaging, plethysmography sensor, pulse oximetry sensor, pulse pressure sensors, inertial measurement unit (IMU), goniometer, insole, electroencephalography (EEG), skin resistance sensor, infrared sensor, spirometer (with or without dilution), stethoscope, tensiometer, intestinal motility sensor, bowel sensor and/or pH sensor.

The use of the neurostimulation system may comprise and/or may be combined with the use of a feedback system, wherein the feedback system is an audio and/or visual and/or sensory-motoric feedback system. The feedback system can provide the subject with feedback information (e.g., concerning the neuromodulation system's performance and/or the patient's performance and/or patient's body reaction(s)). In this manner, the feedback system can support or enhance rehabilitation. In some embodiments, the feedback system can be part of the closed-loop system. In particular, the audio feedback system may include, for example, headphones, speakers, providing sound or voice signals. The visual feedback system may include, for example, floor projection, goggles, LEDs and/or lights, and/or a screen for providing visual signals.

The sensory-motoric feedback system may provide and/or comprise signals such as haptic signals, pain, paresthesia, signals provided by a person, sensory pathways stimulation, temperature signals and/or changes, touch, vibration etc. The feedback system may be part of the closed-loop system and/or vice versa.

The at least one sensor may provide quality and/or performance metrics of the use of the neuromodulation system. The quality and/or performance metrics may be or may comprise sensitivity scores, motor scores, quality of movement scores, performance and/or activity, participation, reaction times, clinical assessment scores, autonomous system scores. In particular, the quality and/or performance scores may be or may comprise autonomous muscular reflexes and/or stretch reflexes, fluidity, blood O2 saturation, blood speed, distance to path ratio, cardiac parameters, gait parameters, heart rate, force, speed, distance, stress level, muscle strength, range of motion, pulse pressure wave, QoL questionnaires, quantity and timing of urine, respiratory rate, respiratory volume, or stress markers (e.g., stress markers in body fluids).

In some embodiments, the neuromodulation system may provide tonic and/or burst and/or high-frequency neuromodulation. Tonic neuromodulation may be applied with 1 to 125 Hz. Burst neuromodulation may be applied by applying pairs, triplets, quadruplets, quintuplets of pulse at burst frequency of at least 200Hz, preferably 300 to 750 Hz, at an interburst frequency between 1 and 125 Hz, preferably 10 to 60Hz. High frequency neuromodulation may be applied with 200 Hz to 10 kHz.

In some embodiments, the neuromodulation system may provide frequency neuromodulation on top of a carrying frequency. In particular, the carrying frequency may be applied with 1 to 10 kHz, preferably 5 to 10 kHz.

In some embodiments, indoor and/or outdoor use of the neuromodulation system may be possible. In some instances, the use of the system may be possible during activities performed by the subject. For example, this may enable use of the neuromodulation system during daily life activities and/or sports (e.g., walking, running, cycling, hiking, horse riding, skiing, swimming, sailing, jumping, ball sports, dancing), etc.

Indoor use may comprise use in least one of clinics, gym, physiotherapy room, occupational room, aquatic room, sport activity room, recreation facility, restaurant, cinema, bathroom, bedroom, kitchen, living room, etc.

Outdoor use may comprise use on streets (including gravel, concrete pavement, cobble stone, rocky hiking trails, etc.), grass, shallow water, sand and/or snow.

It is generally possible that the neuromodulation system may be used and/or operated by at least one robot and/or a person. In particular, the robot may be an assistive robot for healthcare and/or a humanoid robot. In particular, the person may be a family member, a friend, a therapist, a physiotherapist, a nurse, an electrophysiologist, a non-professional user, a professional user, an occupational therapist, a surgeon, a technician (clinical/FCE), and/or a (treating) physician of the subject and/or the subject himself. Further, the neuromodulation system may be used by a virtual assistant. Additionally, and/or alternatively, the neuromodulation system may be operated locally or remotely.

Consistent with disclosed embodiments, the use of the neuromodulation system may comprise the use of a network and/or device and/or platform. In particular, the use of the neuromodulation system may comprise the use of at least one of a cloud, game console, internet, local database, local network, medical and/or rehabilitation device, smartphone, smartwatch and/or social media.

In some embodiments, the use of the neuromodulation system may be performed to perform a task including but not limited to balancing, bending over, coughing, walking, climbing, climbing stairs, swimming and/or performing other tasks under water, sitting, sitting on ground, sit-to-stand, grasping, squatting, standing, swallowing, erecting, inclined waking, neck training, posture training, normal neck posture, normal gait, normal intestinal movement during digestion, normal trunk posture, (normal(use of at least one affected limb, (normal) use of lower limbs, prevention of muscle weakness, regulation of stomach acidity and mucosal layer, respiration, transferring, voluntary regulated sphincter action, zero-G-training and/or performing above-mentioned tasks in zero-G.

The use of the neuromodulation system may be performed to evoke responses such as but not limited to cardiac response, hormonal response, motor response, neurotransmitter response, sensory response, smooth muscle contraction, smooth muscle dilatation, general somatic afferent response (perception of touch, pain and/or temperature and/or others), general somatic efferent response (voluntary motor innervation and/or others), general visceral efferent response (motor innervation to smooth muscles and/or heart muscle and/or glands), special afferent response (visceral: smell, taste, somatic: vision, hearing, balance), respiration, cough and/or erection.

Gastrointestinal System

In some embodiments, a neuromodulation system consistent with disclosed embodiments can be used to treat and/or modulate of at least one condition and/or function and/or dysfunction of the subject's gastrointestinal system. The at least one condition and/or function and/or dysfunction can be or include bowel sphincter function. In various embodiments, the at least one condition and/or function could be or include at least one of acidity of stomach, cholesterol imbalance, lipogenesis/lipolysis imbalance, mucosa renewal, intestinal immobility, lower esophageal sphincter function, salivary glands function, mucous layer of stomach function, swallowing, taste, immune system deficit, lipid metabolism and/or glycogen metabolism. In some embodiments, the at least one abnormal body function and/or disease can be incontinence.

In various embodiments, the abnormal body and/or disease could be or could comprise ulcers, Helicobacter Pylori infection, inflammatory bowel disease, Crohn's disease, ulcerative colitis, irritable bowel syndrome, tumor, gastroesophageal reflux disease, lower esophageal sphincter failure, achalasia, trauma, spinal cord injury, stroke, tumor, pernicious anemia, vitamin D deficiency, Sjogren syndrome, Mikulicz syndrome, dysphagia, diabetes and/or obesity.

Consistent with disclosed embodiments, treating the subject can include treating the subject's lower bowel sphincter. In various embodiments, treating the subject comprises treating a voluntary regulated sphincter of the subject. In various embodiments, treating the subject could be or could comprise treating at least one of stomach, autonomous regulated sphincter, pancreas, dorsal root to muscle (e.g., pharynx), circulatory cholesterol uptake, colon, vago-vagal reflex, cajal cell, smooth muscle, nerve, Auerbach plexus, Meissner plexus, lower esophageal sphincter, liver, pancreas and/or salivary gland.

Consistent with disclosed embodiments, the neuromodulation system can provide treatment throughout rehabilitation of the subject. In various embodiments, neuromodulation system can provide treatment during at least one stage of rehabilitation. The stage of rehabilitation could be or could comprise at least one of stabilization in bed, mobilization in bed, mobilization at bedside, verticalization, pre-surgery, post-surgery, pre-implantation, post-implantation, spinal shock and/or daily life activities.

In some embodiments, the neuromodulation system can provide treatment to evoke or assist voluntary regulated sphincter action. In various embodiments, the neuromodulation system can provide treatment to evoke or assist a task. The task can include at least one of normal intestinal movement during digestion, regulation of stomach acidity and/or mucosal layer, eating, swallowing and/or drinking. Consistent with disclosed embodiments, the neuromodulation system can be used indoors and/or outdoors. Furthermore, the system can be used during activities performed by the subject.

In some embodiments, the neuromodulation system can be a neurostimulation system, wherein the neurostimulation system can include an electrical neurostimulation system. However, alternatively and/or additionally, a mechanical neurostimulation system and/or pharmacological neurostimulation system could be generally possible. In this embodiment, the electrical neurostimulation system can be an intramuscular stimulation system.

In an alternative embodiment, the electrical neurostimulation system could be or could include at least one of a transcutaneous electrical stimulation system, epidural electrical stimulation system, functional electrical stimulation system, dorsal root ganglion stimulation system, subdural stimulation system, optogenetics, optotrodes, patch-clamp, intra-cellular electrodes and/or extra-cellular electrodes.

In some instances, for treating ulcers, Helicobacter pylori infection, Crohn's disease, irritable bowel syndrome and/or pernicious anemia, an electrical stimulation system (e.g., epidural and/or subdural stimulation system) could be used to provide stimulation at least partially to the level of T6-T9 and/or L1-S1 of the subject.

In some instances, for treating incontinence (e.g., after spinal cord injury), an electrical stimulation system (e.g., epidural and/or subdural stimulation system) could be used to provide stimulation at least partially to the level of T10-L2 and/or S2 and/or S4 of the subject.

In some instances, for treating diabetes mellitus, an electrical stimulation system (e.g., epidural and/or subdural stimulation system) could be used to provide stimulation at least partially to the level of T7-T12 and/or T5-T8 of the subject.

In some instances, for treating gastroesophageal reflux disease and/or achalasia, an electrical stimulation system (e.g., epidural and/or subdural stimulation system) could be used to provide stimulation at least partially to N.X.

In some instances, for treating pernicious anemia, vitamin D deficiency, diabetes mellitus, Sjogren syndrome, and/or Mikulicz syndrome the electrical stimulation system (e.g., epidural and/or subdural stimulation system) could be used to provide stimulation at least partially to the level of C1-C4 of the subject.

In some instances, for treating dysphagia (e.g., after spinal cord injury), an electrical stimulation system (e.g., epidural and/or subdural stimulation system) could be used to provide stimulation at least partially to N.X., N.IX and/or the level of C1-C5 of the subject.

In some instances, for use of the neuromodulation system could be performed to evoke responses such as defecation.

Consistent with disclosed embodiments, the neuromodulation system can be an open-loop system, a closed-loop system, or a pseudo-open-loop system. In some embodiments, a sensor of a closed-loop neuromodulation device could be or could comprise at least one of intestinal motility sensor, bowel sensor, pH sensor and/or another sensor.

Consistent with disclosed embodiments, the neuromodulation system can be used post implantation of the neuromodulation system. In various embodiments, the neuromodulation system can be used throughout rehabilitation of the subject and/or during at least one stage of stabilization in bed, mobilization in bed, mobilization at bedside, verticalization, surgery, post-surgery, pre-implantation, post-implantation, spinal shock and/or daily life activities.

Consistent with disclosed embodiments, the neuromodulation system can be used indoors. For example, the indoor use can include use in clinics. In various embodiments, the indoor use could include the use in at least one of gym, physiotherapy room, occupational room, aquatic room, sport activity room, recreation facility, restaurant, cinema, bathroom, bedroom, kitchen, living room, etc.

Consistent with disclosed embodiments, the neuromodulation system could be used outdoors. In some embodiments, the system can be used during activities performed by the subject. In some embodiments, the neuromodulation system can be used or operated by at least one person.

Consistent with disclosed embodiments, the neuromodulation system can be used by the subject. In some embodiments, any other person, such as a family member, a friend, a therapist, a physiotherapist, a nurse, an electrophysiologist, a non-professional user, a professional user, an occupational therapist, a surgeon, a technician (clinical/FCE) and/or a (treating) physician of the subject could use the system. In some embodiments, the neuromodulation system can be operated by a robot.

Reproductive System

Consistent with disclosed embodiments, the use of a neuromodulation for treating a subject with at least one abnormal body function and/or disease comprises the treatment and/or modulation of at least one condition and/or function and/or dysfunction of the subject's reproductive system. In some embodiments, the at least one condition and/or function and/or dysfunction can be erectile dysfunction. In some embodiments, the at least one abnormal body function and/or disease can be impairment of genital plexus after prostate removal.

In some embodiments, the at least one abnormal body function and/or disease can be or include at least one of stroke, trauma, spinal cord injury, tumor, diabetes, Parkinson's disease, Alzheimer's disease and/or neurodegenerative disease. Consistent with disclosed embodiments, treating a subject can include treating at least one of the genital plexus, smooth muscle, or nerves of the subject. In some embodiments, the use of the neuromodulation system can be performed throughout rehabilitation of the subject. In various embodiments, the use of the neuromodulation system can be performed during at least one stage of rehabilitation.

Consistent with disclosed embodiments, the stage of rehabilitation could be or could include at least one of stabilization in bed, mobilization in bed, mobilization at bedside, verticalization, blood pressure lowering, blood pressure rehabilitation, cardiac rehabilitation, standing, stepping, balance training, locomotor training, fluent locomotion, pre-surgery, post-surgery, post-implantation, post-setting of neuromodulation system, spinal shock and/or daily life activities.

Consistent with disclosed embodiments, the neuromodulation system can be a neurostimulation system, wherein the neurostimulation system can include an electrical neurostimulation system. However, in various embodiments, the neurostimulation system can be or include a mechanical neurostimulation system and/or pharmacological neurostimulation system.

Consistent with disclosed embodiments, the electrical neurostimulation system can be an epidural electrical stimulation system. In this embodiment, the epidural electrical stimulation system can be used to provide stimulation at least partially to level L5-S5 of the subject.

In some embodiments, the stimulation system could be alternatively and/or additionally at least one of a transcutaneous electrical stimulation system, functional electrical stimulation system, intramuscular stimulation system, dorsal root ganglion stimulation system, subdural stimulation system, optogenetics, optotrodes, patch-clamp, intra-cellular electrodes and/or extra-cellular electrodes.

Consistent with disclosed embodiments, the use of the neuromodulation system is performed to evoke responses such as erection. In various embodiments, any other type of response, such as motor and/or sensory response of the reproductive system could be generally possible.

Consistent with disclosed embodiments, the neuromodulation system may be an open-loop system. In some embodiments, the neuromodulation system may be a closed-loop system and/or pseudo-open-loop system. Consistent with disclosed embodiments, the use of the neuromodulation system can include the use of burst neuromodulation, in particular, burst neurostimulation. In some embodiments, the burst neuromodulation can be applied by applying pairs, triplets, quadruplets, quintuplets of pulse at burst frequency of at least 200 Hz, preferably 300 to 750 Hz, at an interburst frequency between 1 and 125 Hz, preferably between 10 and 60Hz. In various embodiments, the use of tonic and/or high-frequency neuromodulation, in particular neurostimulation could be generally possible.

Consistent with disclosed embodiments, the system could provide frequency neuromodulation on top of a carrying frequency. For example, the carrying frequency could be applied with 1 to 10 kHz, preferably 5 to 10 kHz.

In some embodiments, the use of the neuromodulation system could include the use of an assistive device. In various embodiments, the neuromodulation system could be used without use of an assistive device.

Urinary System

Consistent with disclosed embodiments, the neuromodulation system can be used for the treatment and/or modulation of at least one condition and/or function and/or dysfunction of a subject's urinary system. In some embodiments, the at least one condition and/or function and/or dysfunction could be bladder sphincter function. For example, the neuromodulation system could be used for the treatment and/or modulation of bladder sphincter dysfunction. In various embodiments, the at least one condition and/or function and/or dysfunction can include at least one of blood volume, mean arterial pressure, vesical sphincter, or other functions, conditions, or dysfunctions suitable for treatment according to disclosed embodiments.

Consistent with disclosed embodiments, the at least one abnormal body function and/or disease can be incontinence. In various embodiments, the at least one abnormal body function and/or disease could be or could comprise hypertension, hypotension, sudden episodic high blood pressure, trauma, spinal cord injury, stroke, tumor, damaged dorsal root, Parkinson's disease, Alzheimer's disease, neurodegenerative disease, overreactive bladder syndrome and/or others.

Consistent with disclosed embodiments, treating the subject can include treating at least one muscle of the subject. In some instances, treating the subject can include treating the lower urinary sphincter of the subject. In some embodiments, the neuromodulation system can be used to treat at least one of sympathetic and/or parasympathetic nervous system, autonomous and/or voluntary regulated sphincters, smooth muscles, nerves, kidney, detrusor, kidney and/or anatomical structures suitable for treatment according to the disclosed embodiments.

Consistent with disclosed embodiments, the use of the neuromodulation system can include the use of the neuromodulation system throughout rehabilitation of the subject. In some embodiments, the use of the neuromodulation system can include the use of a neuromodulation system during at least one stage of rehabilitation.

Consistent with disclosed embodiments, the stage of rehabilitation could be or could comprise at least one of stabilization in bed, mobilization in bed, mobilization at bedside, verticalization, blood pressure lowering, blood pressure rehabilitation, cardiac rehabilitation, standing, stepping, balance training, locomotor training, fluent locomotion, pre-surgery, post-surgery, pre-implantation, post-implantation, post-setting of neuromodulation system, spinal shock and/or daily life activities.

In some embodiments, the neuromodulation system can be a neurostimulation system. The neurostimulation system can be or include at least one of an electrical neurostimulation system, a mechanical neurostimulation system, or a pharmacological neurostimulation system. In some embodiments, the electrical neurostimulation system can be an epidural electrical stimulation system.

Consistent with disclosed embodiments, the epidural electrical stimulation system can be used to provide stimulation at least partially to level L1-L2 and/or S2-S4 of the subject. Alternatively, the epidural electrical stimulation system can be used to provide stimulation at least partially to level L3-L4 of the subject.

Consistent with disclosed embodiments, the neurostimulation system could be or include at least one of a transcutaneous electrical stimulation system, functional electrical stimulation system, intramuscular stimulation system, dorsal root ganglion stimulation system, subdural stimulation system (also for the treatment of overreactive bladder syndrome), optogenetic stimulation system, optotrode stimulation system, patch-clamp, or intra-cellular electrodes or extra-cellular electrode stimulation system.

In some embodiments, for treating hypotension and/or hypertension, the electrical stimulation system (e.g., epidural and/or subdural stimulation system) can be used to provide stimulation at least partially to level T10-L1 of the subject.

Consistent with disclosed embodiments, the neuromodulation system can be used indoors or outdoors. In some embodiments, the use of the system can be possible during activities performed by the subject.

Consistent with disclosed embodiments, the neuromodulation system can be used to provide tonic neuromodulation, in particular tonic neurostimulation. In some instances, tonic neuromodulation could be applied with 1 to 125 Hz. In some embodiments, the neuromodulation system can provide frequency neuromodulation on top of a carrying frequency. In some embodiments, the carrying frequency can be between 1 to 10 kHz, preferably between 5 to 10 kHz. In some embodiments, the neuromodulation system can provide burst and/or high-frequency neuromodulation.

Consistent with disclosed embodiments, the neuromodulation system can be a closed-loop system. In some embodiments, the neuromodulation system can be an open-loop system or a pseudo-open-loop system. Consistent with disclosed embodiments, the closed-loop neuromodulation system can include a sensor. The sensor of such a closed-loop neuromodulation system can be or include at least one of a tensiometer and/or electrocardiogram and/or any other type of sensor.

Consistent with disclosed embodiments, the neuromodulation system can be used or operated by at least one person. The person can be a family member, a friend, a therapist, a physiotherapist, a nurse, an electrophysiologist, a non-professional user, a professional user, an occupational therapist, a surgeon, a technician (clinical/FCE), and/or a (treating) physician of the subject. In some embodiments, the neuromodulation system can be operated by a robot.

Consistent with disclosed embodiments, the neuromodulation system can be used together with an assistive device. In various embodiments, an assistive device need not be used together with the neuromodulation system.

Consistent with disclosed embodiments, the neuromodulation system can be configured to provide neuromodulation stimulation for evoking a response of the sphincter (e.g., a contraction or relaxation).

Cardiovascular System

In some embodiments, a neuromodulation system consistent with disclosed embodiments can be used to treat and/or modulate of at least one condition and/or function and/or dysfunction of a subject's cardiovascular system. The at least one condition and/or function and/or dysfunction can be or include mean venous pressure or mean arterial pressure. In some embodiments, the abnormal body function can be or include hypertonia. In various embodiments, the at least one condition and/or function and/or dysfunction could include at least one of cardiac activity, supraspinal cardiovascular regulation, autonomic dysreflexia, orthostatic hypotension and/or mean venous pressure or mean arterial pressure.

In some embodiments, the abnormal body function and/or disease can be or include dystonia, spinal cord injury, hypotension, sudden episodic high blood pressure, supraspinal cardiovascular regulation, cardiac failure, cardiac dysrhythmia, bradycardia, tachycardia, cardiac arrest, tumor, trauma and/or stroke.

Consistent with disclosed embodiments, treating the subject can include treating nerves of the subject. In various embodiments, treating the subject can include treating the spinal cord of the subject.

Consistent with disclosed embodiments, the treatment of a subject can be performed directly and/or indirectly. In various embodiments, treating the subject can include treating at least one of heart, cardiac muscle, muscle, smooth muscles, nerves, Renin Angiotensin system, kidney, adrenal gland and/or sinoatrial/atrioventricular nodes.

Consistent with disclosed embodiments, the neuromodulation system can be a neurostimulation system. The neurostimulation system can be or include at least one of an electrical neurostimulation system, mechanical neurostimulation system, or pharmacological neurostimulation system.

Consistent with disclosed embodiments, the neurostimulation system can be an epidural electrical stimulation system. In some embodiments, the epidural electrical stimulation system can be used to provide stimulation at least partially to the level of T10-L1 of the subject. In some embodiments, the stimulation system could be alternatively and/or additionally at least one of a transcutaneous electrical stimulation system, functional electrical stimulation system, intramuscular stimulation system, dorsal root ganglion stimulation system, subdural stimulation system, cardiac stimulation system, optogenetics, optotrodes, patch-clamp, intra-cellular electrodes and/or extra-cellular electrodes.

In some embodiments, an electrical stimulation system (e.g., epidural and/or subdural stimulation system) can be used to provide stimulation at least partially to the level of T6 or higher and/or T1-T6 of the subject.

In some embodiments, for treating cardiac failure and/or spinal cord injury, an electrical stimulation system (e.g., epidural and/or subdural stimulation system) could be used to provide stimulation at least partially to the level of T1-T5 of the subject.

In some embodiments, for treating hypertonia and/or spinal cord injury (e.g., when autonomic dysreflexia occurs) the electrical stimulation system (e.g., epidural and/or subdural stimulation system) could be used to provide stimulation at least partially at to level of C1-C4 of the subject and above.

In various embodiments, the neurostimulation system can be a dorsal root ganglion stimulation system and/or subdural stimulation system or cardiac stimulation system.

In certain embodiments, the neuromodulation system can be used post-implantation in the subject. For example, an epidural electrical stimulation system may be used post-implantation in the subject.

Consistent with disclosed embodiments, the neuromodulation system can be used throughout rehabilitation of the subject and/or during at least one stage of rehabilitation. In particular, the neuromodulation system can be used at a rehabilitation stage including at least one of stabilization in bed, mobilization in bed, mobilization at bedside, verticalization, blood pressure lowering, blood pressure rehabilitation, blood pressure stabilization, cardiac rehabilitation, cardiac stabilization standing, stepping, balance training, locomotor training, fluent locomotion, pre-surgery, post-surgery, pre-implantation, post-setting of neuromodulation system, spinal shock and/or daily life activities.

Consistent with disclosed embodiments, the neuromodulation system can be used indoors or outdoors. In some embodiments, the neuromodulation system can be used during activities performed by the subject.

Consistent with disclosed embodiments, the neuromodulation system can perform neuromodulation to modulate smooth muscle contraction (response). In various embodiments, the neuromodulation system can perform neuromodulation to evoke responses such as but not limited to cardiac response, hormonal response, neurotransmitter response, sensory response and/or smooth muscle dilatation.

Consistent with disclosed embodiments, the use of the neuromodulation system can include the use of tonic neuromodulation, in particular, tonic neurostimulation. In some embodiments, the tonic neuromodulation may be applied with 1 to 125 Hz. In some embodiments, the neuromodulation system could provide frequency neuromodulation on top of a carrying frequency. For example, the carrying frequency could be applied with 1 to 10 kHz, preferably 5 to 10 kHz. In various embodiments, the neuromodulation system could provide burst or high-frequency neuromodulation (e.g., neurostimulation).

Consistent with disclosed embodiments, the neuromodulation system can be an open-loop system. In some embodiments, the neuromodulation system can be a closed-loop system or pseudo-open-loop system. In some embodiments, a sensor of such a closed-loop neuromodulation system can be or include at least one of a tensiometer, pulse pressure measuring method, electrocardiogram, pulse oximetry, MRI, doppler ultrasound and/or any other type of sensor. The at least one sensor can provide quality and/or performance metrics of the use of the neuromodulation system. The quality and/or performance metrics can be or include cardiac parameters. In some embodiments, the neuromodulation system can include at least one of a pressure sensor, a rehabilitation device, an electrode, an electrode lead, an electrocardiogram, magnetic resonance imaging, a pulse oximetry sensor, or a pulse pressure sensor.

Consistent with disclosed embodiments, the quality and/or performance metrics could comprise fatigue measures, stress measures (e.g., in body fluids), questionnaires (in particular QoL questionnaires), pulse pressure wave, blood O2 saturation, blood speed, QoL questionnaires, q and/or stress markers (e.g., stress markers in body fluids).

Consistent with disclosed embodiments, the neuromodulation system can provide treatment together with an assistive device. In various embodiments, the neuromodulation system can be used without an assistive device. Such an assistive device can be or include at least one of pulling handles, cables from ceiling, aquatic equipment, a treadmill, an exoskeleton, an orthosis, a robot, a bike, a breather, crutches, a cuff, a cuff for functional electrical stimulation, a smartwatch, a motorized mattress, non-invasive ventilation, a standing frame, a rollator, a body weight support system, elastic bands, weights, a slide board, a step machine, a trike, a walker, a robot-physiotherapist, balls and/or half-balls.

Consistent with disclosed embodiments the neuromodulation system can be used by a (treating) physician. In various embodiments, the neuromodulation system can be used by another person. Such a person could be family member, a friend, a therapist, a physiotherapist, a nurse, an electrophysiologist, a non-professional user, a professional user, an occupational therapist, a surgeon and/or a technician (clinical/FCE) of the subject.

Consistent with disclosed embodiments, the use of the neuromodulation system can include the use of a network device. In some embodiments, the use of the neuromodulation system can include the use of a smartphone. In various embodiments, the use of the neuromodulation system could include the use of a network and/or platform and/or alternative network device. In some embodiments, the use of the neuromodulation system can include the use of at least one of a cloud, game console, internet, local database, local network, medical and/or rehabilitation device, smartwatch and/or social media.

Consistent with disclosed embodiments, the use of the neurostimulation system can include the use of a feedback system. The feedback system can be a visual feedback system. In some embodiments, the feedback system can be a screen. In various embodiments, the feedback system can be or can include an audio feedback system and/or sensory-motoric feedback system. In some instances, the feedback system can be or include headphones and/or haptic signals.

Respiratory System

In some embodiments, a neuromodulation system consistent with disclosed embodiments can be used to treat and/or modulate of at least one condition and/or function and/or dysfunction of a subject's respiratory system. The at least one condition and/or function and/or dysfunction can be or include a breathing deficiency. In some embodiments, at least one abnormal body function can be or include dyspnea. In various embodiments, the at least one condition and/or function and/or dysfunction could include at least one of bronchial tree obstruction and/or bronchial deficiency and/or alveolar deficiency.

In some embodiments, the at least one abnormal body function and/or disease can be or include at least one of chronic obstructive pulmonary disease, tumor, trauma, stroke, damaged dorsal root, spinal cord injury and/or amyotrophic lateral sclerosis (ALS).

Consistent with disclosed embodiments, treating a subject can include treating the intercostal muscles of the subject. In various embodiments, treating the subject can include treating at least one of bronchia, smooth muscles of the pulmonary system, nerve, alveoli, muscles, solitary tract, pulmonary circulation and/or diaphragm.

Consistent with disclosed embodiments, the neuromodulation system can be a neurostimulation system. The neurostimulation system can be or include at least one of an electrical neurostimulation system, mechanical neurostimulation system, or pharmacological neurostimulation system.

Consistent with disclosed embodiments, the neurostimulation system can be an intramuscular stimulation system. In various embodiments, the neurostimulation system can be a dorsal root ganglion stimulation system and/or subdural stimulation system and/or transcutaneous electrical stimulation system and/or epidural electrical stimulation system.

In some embodiments, for treating dyspnea and/or spinal cord injury (e.g., for treating dyspnea after spinal cord injury) and/or chronic obstructive pulmonary disease, the electrical stimulation system (e.g., epidural and/or subdural stimulation system) can be used to provide stimulation at least partially to the level of C3-C5 of the subject.

In some embodiments, for treating ALS (e.g., breathing deficiency caused by ALS), the electrical stimulation system (e.g., epidural and/or subdural stimulation system) can be used to provide stimulation at least partially to the level of T1-T6 of the subject.

In some embodiments, the neuromodulation system can be used post-implantation in the subject, in particular post implantation of the intramuscular stimulation system.

In various embodiments, the use of the neuromodulation system can include the use of the neuromodulation system throughout rehabilitation of the subject and/or during at least one stage of stabilization in bed, mobilization in bed, mobilization at bedside, verticalization, stabilization of blood pressure, standing, stepping, balance training, locomotor training, fluent locomotion, pre-surgery, post-surgery, pre-implantation, post-implantation, post-setting of neuromodulation system, spinal shock and/or daily life activities.

Consistent with disclosed embodiments, the system can be used indoors. In some embodiments, such indoor use can include use in least one of clinics, gym, physiotherapy room, occupational room, aquatic room, sport activity room, recreation facility, restaurant, cinema, bathroom, bedroom, kitchen, living room, or similar indoor environments. In various embodiments, the neuromodulation system can be used outdoors. Consistent with disclosed embodiments, the neuromodulation system can be used during activities performed by the subject.

Consistent with disclosed embodiments, the neuromodulation system can be used together with an assistive device. In various embodiments, an assistive device need not be used together with the neuromodulation system.

Consistent with disclosed embodiments, the neuromodulation system can be configured to provide tonic neuromodulation (e.g., tonic neurostimulation). In some embodiments, tonic neuromodulation could be applied at frequencies between 1 and 125 Hz.

Consistent with disclosed embodiments, the neuromodulation system can provide frequency neuromodulation on top of a carrying frequency. In particular, the carrying frequency can be applied with 1 to 10 kHz, preferably 5 to 10 kHz. In some embodiments, the neuromodulation system can provide burst and/or high-frequency neuromodulation (e.g., neurostimulation).

Consistent with disclosed embodiments, the neuromodulation system can be a closed-loop system. In some embodiments, the neuromodulation system can be an open-loop system or a pseudo-open-loop system. In some embodiments, the closed-loop system can include one or more sensors. The one or more sensors can be or include at lease one of a plethysmography sensor, a thermometer (e.g., a nose thermometer), a pressure sensor, a pulse oximetry sensor, an inertial measurement unit (IMU), a goniometer, an insole, an electroencephalography sensor (EEG), an electromyography sensor (EMG), or another suitable sensor.

Consistent with disclosed embodiments, the at least one sensor can provide quality and/or performance metrics of the use of the neuromodulation system. In some embodiments, the quality and/or performance metric can be a respiratory rate. In various embodiments, the quality and/or performance metrics can be or include at least one of respiratory volume, blood O2 saturation, or stress markers (e.g., stress markers measured in body fluids).

Consistent with disclosed embodiments, the neuromodulation system can be used or operated by at least one person. In some embodiments, the neuromodulation system can be used by the subject; or by at least one of a family member, a friend, a therapist, a physiotherapist, a nurse, an electrophysiologist, a non-professional user, a professional user, an occupational therapist, a surgeon, a technician (clinical/FCE), and/or a (treating) physician of the subject.

Consistent with disclosed embodiments, the neuromodulation system can provide neuromodulation configured to evoke a response. The response can be at least one of respiration or a cough.

Sensory System

Consistent with disclosed embodiments, the neuromodulation system can be used for the treatment and/or modulation of at least one condition and/or function and/or dysfunction of a subject's sensory system. In some embodiments, the at least one condition and/or function and/or dysfunction could be or include at least one of back pain, odor, or taste.

Consistent with disclosed embodiments, the at least one abnormal body function and/or disease can be trauma. In various embodiments, the at least one abnormal body function and/or disease can be or include at least one of a tumor, a herniated disk, Parkinson's disease, Alzheimer's disease, a neurodegenerative disease, a stroke, or spinal cord injury.

Consistent with disclosed embodiments, treating the subject can include treating at least one nerve of the subject. The treatment of the subject can be performed directly and/or indirectly.

Consistent with disclosed embodiments, the neuromodulation system can be a neurostimulation system. The neurostimulation system can be or include at least one of an electrical neurostimulation system, a mechanical neurostimulation system, or a pharmacological neurostimulation system.

Consistent with disclosed embodiments, the neurostimulation system can be an epidural electrical stimulation system. In some embodiments, the epidural electrical stimulation system can be used to provide stimulation at least partially to the level of T2-L1 of the subject. In various embodiments, the epidural electrical stimulation system can be used to provide stimulation at least partially above C1 of the subject.

Consistent with disclosed embodiments, an electrical neurostimulation system can be or include at least one of transcutaneous electrical stimulation system, dorsal root ganglion stimulation system and/or subdural stimulation system.

Consistent with disclosed embodiments, the neuromodulation system can be used post-implantation in a subject. For example, an epidural electrical stimulation system can be used post implantation in the subject. In various embodiments, the neuromodulation system can be used throughout rehabilitation of the subject and/or during at least one of stabilization in bed, mobilization in bed, mobilization at bedside, verticalization, blood pressure lowering, blood pressure rehabilitation, blood pressure stabilization, cardiac rehabilitation, cardiac stabilization, standing, stepping, balance training, locomotor training, fluent locomotion, pre-surgery, post-surgery, pre-implantation, post-setting of neuromodulation system, spinal shock and/or daily life activities.

Consistent with disclosed embodiments, the neuromodulation system can be a closed-loop system. In some embodiments, the neuromodulation system can be an open-loop system or a pseudo-open-loop system. Consistent with disclosed embodiments, the closed-loop neuromodulation system can include a sensor.

Consistent with disclosed embodiments, the neuromodulation system can be configured to use burst neuromodulation (e.g., burst neurostimulation). In some embodiments, the neuromodulation system can be configured to apply burst neuromodulation comprising pairs, triplets, quadruplets, quintuplets of pulses. The pulses can have a burst frequency of at least 200 Hz, preferably 300 to 750 Hz. The pulses can have an interburst frequency of between 1 and 125 Hz, preferably between 10 and 60 Hz.

Consistent with disclosed embodiments, the neuromodulation system can be configured to provide frequency neuromodulation on top of a carrying frequency. In particular, the carrying frequency could be applied at between 1 and 10 kHz, preferably between 5 and 10 kHz. In various embodiments, the neuromodulation system can be configured to use of tonic and/or high-frequency neuromodulation (e.g., neurostimulation).

Consistent with disclosed embodiments, the neuromodulation system can be used indoors or outdoors. In some embodiments, the neuromodulation system can be adapted for use in a clinic, gym, physiotherapy room, occupational room, aquatic room, sport activity room, recreation facility, restaurant, cinema, bathroom, bedroom, kitchen, living room, or other suitable room. In some embodiments, the use of the system can be possible during activities performed by the subject.

Consistent with disclosed embodiments, the neuromodulation system can be used or operated by at least one person. The person can be the subject; or at least one of a family member, a friend, a therapist, a physiotherapist, a nurse, an electrophysiologist, a non-professional user, a professional user, an occupational therapist, a surgeon, a technician (clinical/FCE), and/or a (treating) physician of the subject.

Locomotor System

Consistent with disclosed embodiments, the neuromodulation system can be used for the treatment and/or modulation of at least one condition and/or function and/or dysfunction of a subject's locomotor system. In some embodiments, the at least one condition and/or function and/or dysfunction could be or include a gait deficit. In various embodiments, the at least one condition and/or function and/or dysfunction can include at least one of individual limb paralysis, lower limb paralysis, upper limb paralysis, neck stabilization and/or trunk stabilization, or other functions, conditions, or dysfunctions suitable for treatment according to disclosed embodiments.

Consistent with disclosed embodiments, the at least one abnormal body function and/or disease can be spinal cord injury. In various embodiments, the at least one abnormal body function or disease can be or include at least one of epilepsy, muscular hyperfatigability, herniated disk, tumor, amyotrophic lateral sclerosis, multiple sclerosis, trauma, stroke, damaged dorsal root, autoimmune disease, loss of sympathetic control, neurodegenerative disease, Parkinson's disease, essential tremor, Alzheimer's disease and/or osteoporosis.

Consistent with disclosed embodiments, treating the subject can include treating the spinal cord of the subject. In various embodiments, treating a subject can include treating at least one of muscular cholinergic synapse, sympathetic and/or parasympathetic nervous system, spinal cord, dorsal root to muscle, muscle, or smooth muscle and/or nerve.

Consistent with disclosed embodiments, the neuromodulation system can be used during locomotor training of the subject. In various embodiments, the neuromodulation system could be used throughout rehabilitation of the subject and/or during at least one of mobilization in bed, mobilization at bedside, verticalization, stepping, balance training, fluent locomotion, pre-surgery, post-surgery, pre-implantation, post-implantation, post-setting of neuromodulation system, spinal shock and/or daily life activities.

Consistent with disclosed embodiments, the neuromodulation system can be used together with an assistive device. For example, use of the neuromodulation system can include the use of an assistive device or be combined with the use of the assistive device. In some embodiments, the assistive device can be an exoskeleton. In various embodiments, the assistive device can be or include at least one of pulling handles, cables from ceiling, aquatic equipment, a treadmill, an orthosis, a robot, a bike, crutches, a cuff, a cuff for functional electrical stimulation, a smartwatch, a motorized mattress, a standing frame, a rollator, a body weight support system, elastic bands, weights, a slide board, a step machine, a trike, a walker, a robot-physiotherapist, balls and/or half-balls.

Consistent with disclosed embodiments, the neuromodulation system can be configured to provide neuromodulation stimulation to evoke or assist a normal gait of a subject. In various embodiments neuromodulation system can be configured to provide neuromodulation stimulation to evoke or assist a task of the subject including at least one of balancing, bending over, walking, climbing, climbing stairs, swimming and/or performing other tasks under water, sitting, sitting on ground, sit-to-stand, grasping, squatting, standing, inclined waking, neck training, posture training, normal neck posture, normal trunk posture, normal use of at least one affected limb, normal use of lower limbs, prevention of muscle weakness, or training with body weight support and/or performing above-mentioned tasks with body weight support.

Consistent with disclosed embodiments, the neuromodulation system can be or include a neurostimulation system. The neurostimulation system can be or include at least one of an electrical neurostimulation system, a mechanical neurostimulation system, or a pharmacological neurostimulation system. In some embodiments, an electrical neurostimulation system can be an epidural electrical stimulation system. In various embodiments, the electrical neurostimulation system can be or include at least one of transcutaneous electrical stimulation system, dorsal root ganglion stimulation system, intramuscular stimulation system and/or subdural stimulation system.

Consistent with disclosed embodiments, the electrical stimulation system can be used to provide stimulation at least partially to the level of L2-S1 of the subject. In various embodiments, the electrical stimulation system can be used to provide stimulation at least partially to the level of L2-S2 of the subject.

Consistent with disclosed embodiments, when the neuromodulation system is used for neck stabilization, the electrical stimulation system can be used to provide stimulation at least partially to the level of T2-L1, preferably to the level of T7-L1 of the subject.

Consistent with disclosed embodiments, when the neuromodulation system is used for treating upper and/or lower limb paralysis, the electrical stimulation system can be used to provide stimulation at least partially to the level of C1-S1 of the subject.

Consistent with disclosed embodiments, the neuromodulation system can be a closed-loop system. In some embodiments, the neuromodulation system can be an open-loop system or a pseudo-open-loop system. Consistent with disclosed embodiments, the closed-loop neuromodulation system can include a sensor. The sensor of such a closed-loop neuromodulation system can be or include at least one of an IMU, accelerometer, pressure sensor, motion capture sensor, force plate, rehabilitation device, electrode, electrode lead, infrared camera, GPS, smartphone, smartwatch, action-camcorder, video processing, virtual reality module, magnetic resonance imaging, goniometer, insole, EEG, skin resistance sensor, or infrared sensor.

Consistent with disclosed embodiments, the use of the neuromodulation system can include the use of tonic neuromodulation (e.g., tonic neurostimulation). In some instances, tonic neuromodulation can be applied at a frequency between 1 and 125 Hz. In some embodiments, the system can provide frequency neuromodulation on top of a carrying frequency. In particular, a frequency of the carrying frequency can be between 1 and 10 kHz, preferably between 5 and 10 kHz. In various embodiments, the neuromodulation system can be configured to use burst and/or high-frequency neuromodulation (e.g., neurostimulation).

Consistent with disclosed embodiments, the neuromodulation system can be used post-implantation of the neuromodulation system. In various embodiments, the neuromodulation system can be used throughout rehabilitation of the subject and/or during at least one of stabilization in bed, mobilization in bed, mobilization at bedside, standing, stepping, balance training, locomotor training, fluent locomotion, pre-surgery, post-surgery, post-setting of neuromodulation system, spinal shock and/or daily life activities.

Consistent with disclosed embodiments, the system can be used indoors. In some embodiments, such indoor use can include use in least one of clinics, gym, physiotherapy room, occupational room, aquatic room, sport activity room, recreation facility, restaurant, cinema, bathroom, bedroom, kitchen, living room, or similar indoor environments. In various embodiments, the neuromodulation system can be used outdoors. Consistent with disclosed embodiments, the neuromodulation system can be used during activities performed by the subject.

Consistent with disclosed embodiments, the neuromodulation system can be used or operated by at least one person. In some embodiments, the neuromodulation system can be used by the subject; or by at least one of a family member, a friend, a therapist, a physiotherapist, a nurse, an electrophysiologist, a non-professional user, a professional user, an occupational therapist, a surgeon, a technician (clinical/FCE), and/or a (treating) physician of the subject.

Thermoregulation System

Consistent with disclosed embodiments, the neuromodulation system can be used for the treatment and/or modulation of at least one condition and/or function and/or dysfunction of a subject's thermoregulation system. In some embodiments, the at least one condition and/or function and/or dysfunction can include cutaneous vascularization. In various embodiments, the at least one condition could additionally and/or alternatively include at least one of blood volume, thermoregulation, lipogenesis/lipolysis imbalance, sweat glands, or lipid metabolism.

Consistent with disclosed embodiments, the at least one abnormal body function and/or disease can be spinal cord injury. In various embodiments, the at least one abnormal body function and/or disease can be or include at least one of tumor, obesity, trauma, stroke, damaged dorsal root, or hyperthyroidism.

Consistent with disclosed embodiments, treating the subject can include treating the spinal cord of the subject. In various embodiments, treating the subject can include treating at least one of sympathetic and/or parasympathetic nervous system, dorsal root to muscle, muscle, smooth muscle and/or nerve, preferably in the brain (e.g., in the thalamus and/or hypothalamus) of the subject.

In some embodiments, the neuromodulation system can be used during spinal shock. In various embodiments, the neuromodulation system can be used throughout rehabilitation of the subject and/or during at least one of stabilization in bed, mobilization in bed, mobilization at bedside, verticalization, blood pressure lowering, blood pressure rehabilitation, blood pressure stabilization, cardiac rehabilitation, cardiac stabilization, standing, stepping, balance training, locomotor training, fluent locomotion, pre-surgery, post-surgery, pre-implantation, post-implantation, post-setting of neuromodulation system and/or daily life activities.

Consistent with disclosed embodiments, the neuromodulation system can be configured to use together with an assistive device.

Consistent with disclosed embodiments, neuromodulation system can include a neurostimulation system. The neurostimulation system can be or include at least one of an electrical neurostimulation system, a mechanical neurostimulation system, or a pharmacological neurostimulation system. In some embodiments, an electrical neurostimulation system can include an epidural electrical stimulation system. In various embodiments, the electrical neurostimulation system can be or include at least one of transcutaneous electrical stimulation system, dorsal root ganglion stimulation system, intramuscular stimulation system, or subdural stimulation system.

Consistent with disclosed embodiments, the electrical stimulation system can be used to provide stimulation at least partially to the level of T7 of the subject. In various embodiments, the electrical stimulation system can be used to provide stimulation at least partially to the brain of the subject (e.g., to the hypothalamus of the subject).

Consistent with disclosed embodiments, the neuromodulation system can be a closed-loop system. In some embodiments, the neuromodulation system can be an open-loop system or a pseudo-open-loop system. Consistent with disclosed embodiments, the closed-loop neuromodulation system can include a sensor. The sensor can be or include at least one of a thermometer (e.g., a skin thermometer or implanted thermometer), an electrode, electrode lead, or other type of sensor.

Consistent with disclosed embodiments, the neuromodulation system can be configured to use burst neuromodulation (e.g., burst neurostimulation). In some embodiments, the neuromodulation system can be configured to apply burst neuromodulation comprising pairs, triplets, quadruplets, quintuplets of pulses. The pulses can have a burst frequency of at least 200 Hz, preferably 300 to 750 Hz. The pulses can have an interburst frequency of between 1 and 125 Hz, preferably between 10 and 60 Hz.

Consistent with disclosed embodiments, the use of the neuromodulation system can include the use of tonic neuromodulation (e.g., tonic neurostimulation). In some embodiments, the system can provide frequency neuromodulation on top of a carrying frequency.

Consistent with disclosed embodiments, the system can be used indoors. In some embodiments, such indoor use can include use in least one of clinics, gym, physiotherapy room, occupational room, aquatic room, sport activity room, recreation facility, restaurant, cinema, bathroom, bedroom, kitchen, living room, or similar indoor environments. In various embodiments, the neuromodulation system can be used outdoors. Consistent with disclosed embodiments, the neuromodulation system can be used during activities performed by the subject.

Consistent with disclosed embodiments, the neuromodulation system can be used or operated by at least one person. In some embodiments, the neuromodulation system can be used by the subject; or by at least one of a family member, a friend, a therapist, a physiotherapist, a nurse, an electrophysiologist, a non-professional user, a professional user, an occupational therapist, a surgeon, a technician (clinical/FCE), and/or a (treating) physician of the subject.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

It should be noted that, the relational terms herein such as “first” and “second” are used only to differentiate an entity or operation from another entity or operation, and do not require or imply any actual relationship or sequence between these entities or operations. Moreover, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the terms “and/or” and “or” encompass all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above-described embodiments can be implemented by hardware, or software (program codes), or a combination of hardware and software. If implemented by software, it may be stored in the above-described computer-readable media. The software, when executed by the processor can perform the disclosed methods. The computing units and other functional units described in this disclosure can be implemented by hardware, or software, or a combination of hardware and software. One of ordinary skill in the art will also understand that multiple ones of the above-described modules/units may be combined as one module/unit, and each of the above-described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only, with a true scope and spirit of the invention being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A method for treating a subject with at least one abnormal body function and/or disease, the method comprising the treatment and/or modulation of at least one condition and/or function and/or dysfunction, using a neuromodulation system, of the subject's gastrointestinal system, cardiovascular system, sensory system, urinary system, respiratory system, reproductive system, thermoregulation system and/or locomotor system.

2. The method of claim 1, wherein the at least one condition and/or function and/or dysfunction comprises at least one of acetylcholine dysregulation, acidity of stomach, autonomic dysreflexia, back pain, sphincter function, blood volume dysregulation, breathing deficiency, bronchial tree obstruction, cardiac activity, cholesterol imbalance, motor deficits, mucosa renewal, immune system deficit, immobility of the stomach and/or intestine and/or colon, lipogenesis/lipolysis imbalance, lower limb paralysis and/or upper limb paralysis (bilateral and/or unilateral), detrusor muscle, bronchial deficiency, alveolar deficiency, mean venous pressure, mean arterial pressure, mucous layer of stomach, erectile dysfunction, neck stabilization, odor, hypotension, orthostatic hypotension, hypertension, salivary glands function, supraspinal cardiovascular regulation, swallowing, taste, thermoregulation, trunk stabilization, vision, cutaneous vascularization, sweat glands, lipid metabolism and/or glycogen metabolism.

3. The method of claim 1, wherein the at least one abnormal body function and/or disease is or comprises myasthenia, dystonia, epilepsy, muscular hyperfatigability, ulcers, Helicobacter Pylori infection, inflammatory bowel disease, Crohn's disease, ulcerative colitis, irritable bowel syndrome, spinal cord injury, incontinence, hypertension, hypotension, sudden episodic high blood pressure, dyspnea, chronic obstructive pulmonary disease, cardiac failure, cardiac dysrhythmia, bradycardia, tachycardia, cardiac arrest, impairment of genital plexus (e.g. after prostate removal), HIV infection, herniated disk, tumor, obesity, gastroesophageal reflux disease, lower esophageal sphincter failure, achalasia, amyotrophic lateral sclerosis, trauma, stroke, damaged dorsal root, pernicious anemia, multiple sclerosis, diabetes mellitus, autoimmune disease, Sjogren syndrome, Mikulicz syndrome, loss of sympathetic control, dysphagia, overreactive bladder syndrome, neurodegenerative disease, Parkinson's disease, Alzheimer's disease, essential tremor, schizophrenia and/or osteoporosis.

4. The method of claim 1, wherein treating a subject is or comprises treating of at least one of muscular cholinergic synapse, stomach, sympathetic and/or parasympathetic nervous system, heart, cardiac muscle, spinal cord, lower urinary sphincter, autonomous and/or voluntary regulated sphincters, Renin Angiotensin system, kidney, lower bowel sphincter, diaphragm, intercostal muscle, bronchia, smooth muscles of the pulmonary system, solitary tract, sinoatrial/atrioventricular nodes, pancreas, genital plexus, dorsal root to muscle (e.g. pharynx), colon, vago-vagal reflex, cajal cell, muscle, smooth muscle, nerve, concerned nerve or group of nerves, concerned plexus (including but not limited to genital, Auerbach, Meissner, cardiac, pulmonary, cervical, lumbar, sacral and/or celiac plexus), liver, adrenal gland, lower esophageal sphincter, alveoli, pulmonary circulation, associated cortex and/or regulatory center, salivary gland and/or detrusor muscle.

5. The method of claim 1, wherein treatment occurs throughout rehabilitation of the subject and/or during at least one stage of rehabilitation, wherein the stage of rehabilitation is or comprises at least one of stabilization in bed, mobilization in bed, mobilization at bedside, verticalization, blood pressure lowering, blood pressure rehabilitation, blood pressure stabilization, cardiac rehabilitation, cardiac stabilization, standing, stepping, balance training, locomotor training, fluent locomotion, pre-surgery, post-surgery, pre-implantation, post-implantation, post-setting of neuromodulation system, spinal shock and/or daily life activities.

6. The method of claim 1, wherein the use of the neuromodulation system comprises or is combined with the use of an assistive device, wherein the assistive device is or comprises at least one of a breathing device, pulling handles, cables from ceiling, aquatic equipment, a treadmill, an exoskeleton, an orthosis, a robot, a bike, a breather, crutches, a cuff, a cuff for functional electrical stimulation, a smartwatch, a motorized mattress, non-invasive ventilation, a standing frame, a rollator, body-weight support system, elastic bands, weights, a slide board, a step machine, a trike, a walker, a robot-physiotherapist, balls and/or half-balls.

7. The method of claim 1, wherein the neuromodulation system is a neurostimulation system, wherein the neurostimulation system is or comprises an electrical neurostimulation system and/or mechanical neurostimulation system and/or pharmacological neurostimulation system.

8. The method of claim 1, wherein the electrical neurostimulation system is at least one of a transcutaneous electrical stimulation system, epidural electrical stimulation system, functional electrical stimulation system, intramuscular stimulation system, dorsal root ganglion stimulation system, subdural stimulation system, cardiac stimulation system, optogenetics, optotrodes, patch-clamp, intra-cellular electrodes and/or extra-cellular electrodes.

9. The method of claim 7, wherein the system is an open-loop system and/or closed-loop system and/or pseudo-open-loop system.

10. The method of claim 9, wherein the closed-loop system comprises at least one sensor.

11. The method of claim 1, wherein the use of the neurostimulation system comprises and/or is combined with the use of a feedback system, wherein the feedback system is an audio and/or visual and/or sensory-motoric feedback system.

12. The method of claim 1, wherein system provides tonic and/or burst and/or high-frequency neuromodulation.

13. The method of claim 1, wherein the system provides frequency neuromodulation on top of a carrying frequency.
